(19) United States
(12) Patent Application Publication
(10) Pub. No.: US 2023/0181681 A1
(43) Pub. Date: Jun. 15, 2023

(54) PEPTIDE FOR SUPPRESSING CORONAVIRUS AND USE THEREOF

(71) Applicant: Industry Academic Cooperation Foundation, Hallym University, Chuncheon-si (KR)

(72) Inventors: Hyung Joo KWON, Cheongju-si (KR); Byoung Kwon PARK, Chuncheon-si (KR); Dong Bum KIM, Chuncheon-si (KR); Jin Soo KIM, Seoul (KR)

(21) Appl. No.: 17/922,964

(22) PCT Filed: May 3, 2021
(86) PCT No.: PCT/KR2021/005563

§ 371 Date: Nov. 2, 2022

(30) Foreign Application Priority Data
May 8, 2020 (KR) 10-2020-0055190

(51) Int. Cl. A61K 38/16; C07K 7/08; C07K 14/00; C07K 14/165; A61P 31/14; A61K 47/62

(52) U.S. Cl. CPC A61K 38/162; C07K 7/08; C07K 14/001; C07K 14/165; A61P 31/14; A61K 47/62

(57) ABSTRACT

The present invention relates to: a therapeutic composition for coronavirus comprising, as an active ingredient, one peptide selected from the group consisting of SEQ ID NO: 1, SEQ ID NO: 6, and SEQ ID NO: 8 that binds to a coronavirus N-protein, a coronavirus-derived spike protein, or a fragment of the spike protein; and a composition that binds to a coronavirus N-protein comprising, as an active ingredient, the coronavirus-derived spike protein or the fragment of the spike protein. It is suggested that the peptides of the present invention, based on the understanding and targeting of the interaction of the coronavirus S protein and N protein of the present invention, have an effect that can be helpful in the treatment of coronaviruses including MERS-CoV, SARS-CoV-2, SARS-CoV, and HCoV-OC43.

### TECHNICAL FIELD

The present invention relates to a peptide for suppressing coronavirus and use thereof.

### BACKGROUND ART

Coronavirus is a viral species belonging to the subfamily Coronavirinae of the Coronaviridae family and is a positive-sense RNA virus that infects humans and animals and causes respiratory, gastrointestinal or nervous system diseases.

Coronaviruses occur in animals and can cause severe epidemics in humans, exemplified by Severe Acute Respiratory Syndrome Coronavirus (SARS-CoV) from 2002 to 2003 and Middle East Respiratory Syndrome Coronavirus (MERS-CoV) from 2012, which was confirmed as a novel virus. Surprisingly, these viruses can replicate in the cytoplasm of macrophages, which act in innate immunity, which is important for detecting and eliminating invasive pathogens. Coronaviruses function to interfere with and delay the activation of type I interferon (IFN) and interferon-stimulating genes (ISG) and encode several interferon antagonists for which the expression of a cluster of antagonists contributes to the pathogenesis. A recent study using SARS-CoV infection in mice documented delayed and limited production of interferon that contributes to disease.

Existing vaccine approaches for coronavirus disease are based on spontaneous and natural attenuation, viral inactivation and recombinant viral structural proteins via expression vectors. Existing vaccine candidates do not elicit a strong protective immune response. This lack of long-term protection may be due to the inefficient induction of innate immune responses such as type 1 interferon, a molecule important for promoting adaptive immunity and immune memory.

Meanwhile, Middle East Respiratory Syndrome (MERS) first occurred in the Middle East in September 2012, and according to the WHO announcement, it has spread to 27 countries as of March 2020 and it is a high-risk disease with a fatality rate of 34%, causing 2,494 confirmed cases and 858 deaths.

In addition, in December 2019, a new Severe acute respiratory syndrome coronavirus 2 (SARS-CoV-2), high-risk disease called Coronavirus Infectious Disease-19 (COVID-19) occurred. in Wuhan, China and infected humans causing 2,160,207 confirmed cases and 146,088 deaths worldwide by Apr. 18, 2020.

Middle east respiratory syndrome coronavirus (MERS-CoV), a pathogen of MERS, is a beta-coronavirus (13) newly discovered in 2012 and is a 30,000 kb (+)-sense single-stranded RNA virus and it is a virus similar to the severe acute respiratory syndrome (SARS) virus.

MERS-CoV binds to human DPP4 (Dipeptidyl peptidase 4) receptor using the spike protein and penetrates the cell (Wang N, Shi X, Jiang L J, Zhang S, Wang D, Tong P, Guo D, et al., Cell Research 2013:23:986). MERS-CoV has an incubation period of about one week and causes severe respiratory symptoms such as high fever, cough, and shortness of breath.

Currently, MERS treatment is treated using interferon, an immunomodulatory factor, and ribavirin or lopinavir, an antiviral agent (Public Health England, ISARIC, 2015 Sep. 5 ver 30; Yongpil Jung, Junyoung Song, Yubin Seo et al. al., Infection & Chemotherapy 2015).

Interferon is an immune protein that is released from the body when a virus or bacteria enters the body. It induces the surrounding cells to release antiviral cytokines to suppress virus proliferation and recruits immune cells to remove virus-infected cells. However, the use of interferon causes side effects such as bone marrow dysfunction, anemia, a decrease in the number of white blood cells or a decrease in the number of platelets.

Another therapeutic agent, ribavirin, in the form of a nucleoside analogue, inhibits the proliferation of various viruses by interfering with RNA synthesis. However, ribavirin causes side effects such as toxicity, carcinogenesis, or hemolytic anemia. In addition, the clinical evidence for the administration of interferon and ribavirin for the treatment of MERS is sparse. In the rhesus monkey animal model, a combination of interferon and ribavirin showed a therapeutic effect on MERS (Falzarano D, Wit E, Rasmussen A L et al., Nature Medicine, 2013, 19, 10, p 1313-1318), but actually in the Middle East, a mixture of interferon and ribavirin did not show a significant effect when injected into a total of 5 patients with severe MERS (Al-Tawfiq J A, Momattin H, Dib J et al., International Journal of Infectious Diseases 2014, 20, p 42-46).

So far, the exact cause of the outbreak of coronavirus infection-19 caused by SARS-CoV-2 infection and a treatment for coronavirus infection-19 have not been suggested.

Therefore, there is a need to develop a biologically safe therapeutic agent that can be used for severe patients, immunocompromised patients, and patients with underlying diseases while effectively preventing the proliferation of viruses.

As a related patent, Korean Patent Registration No. 10-1593641 discloses a monoclonal antibody that specifically binds to MERS-CoV nucleocapsid and a composition for diagnosis of MERS-CoV comprising the same.

### DISCLOSURE

### Technical Problem

The present invention was developed in response to the above needs, and an object of the present invention is to provide a novel coronavirus therapeutic agent.

### Technical Solution

To achieve the above object, the present invention provides one peptide selected from the group consisting of SEQ ID NO: 1, SEQ ID NO: 6 and SEQ ID NO: 8 that binds to the coronavirus N-protein.

In one embodiment of the present invention, the peptide is preferably the peptide of SEQ ID NO: 4 but is not limited thereto.

In another embodiment of the present invention, the coronavirus is preferably Severe acute respiratory syndrome coronavirus, Middle East respiratory syndrome coronavirus, Severe acute respiratory syndrome coronavirus 2, or human coronavirus OC43 (HCoV-OC43), but is not limited thereto.

In another embodiment of the present invention, the peptide preferably further comprises a peptide for cell penetration but is not limited thereto.

In one embodiment of the present invention, the cell penetration peptide is preferably a peptide selected from the group consisting of HIV Tat peptide; Pep-1 peptide; oligo-lysine; oligoarginine; and a mixed peptide of oligo-lysine and arginine and in one embodiment of the present invention, the cell penetration peptide is more preferably a D-arginine peptide but is not limited thereto.

In addition, the present invention provides a composition for treatment of a coronavirus comprising a coronavirus-derived spike protein or a spike protein fragment thereof as an active ingredient.

In one embodiment of the present invention, the spike protein fragment is preferably a domain after transmembrane of the spike protein (referred to as ‘C-terminal domain’ or ‘CD’ in the present invention) but is not limited thereto.

The coronavirus-derived spike protein or spike protein fragment thereof is preferably a protein of SEQ ID NO: 1, SEQ ID NO: 4, SEQ ID NO: 6, or SEQ ID NO: 8 or a fragment thereof, but is not limited thereto.

In another embodiment of the present invention, the coronavirus is preferably Severe acute respiratory syndrome coronavirus, Middle East respiratory syndrome coronavirus, Severe acute respiratory syndrome coronavirus 2, or human coronavirus OC43 (HCoV-OC43), but is not limited thereto.

In another embodiment of the present invention, the peptide preferably further comprises a peptide for cell penetration but is not limited thereto.

In one embodiment of the present invention, the cell penetration peptide is preferably a peptide selected from the group consisting of HIV Tat peptide; Pep-1 peptide; oligo-lysine; oligoarginine; and a mixed peptide of oligo-lysine and arginine and in one embodiment of the present invention, the cell penetration peptide is more preferably a D-arginine peptide but is not limited thereto.

The pharmaceutical composition of the present invention may further include a pharmaceutically acceptable carrier, excipient or diluent. Pharmaceutically acceptable carriers, excipients or diluents that can be used in the present invention are not particularly limited as long as they do not impair the effects of the present invention, and include, for example, fillers, extenders, binders, wetting agents, disintegrants, surfactants, lubricants, sweetening agents, flavoring agents, preservatives, and the like. Representative examples of pharmaceutically acceptable carriers, excipients or diluents include lactose, dextrose, sucrose, sorbitol, mannitol, xylitol, maltitol, starch, gelatin, glycerin, acacia gum, alginate, calcium phosphate, calcium carbonate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, propylene glycol, polyethylene glycol, vegetable oil, injectable ester, Witepsol, Macrogol, Tween 61, cacao butter, laurate, etc. are mentioned. The pharmaceutical composition for enhancing innate immunity and antiviral of the present invention may be in a form selected from the group consisting of tablets, pills, powders, granules, capsules, suspensions, emulsions, syrups, aerosols, external preparations, suppositories, and injections. The method for formulating the pharmaceutical composition may be carried out according to a conventional method known in the art and is not particularly limited.

The pharmaceutical composition of the present invention may be administered orally or parenterally, and the dosage may be appropriately selected according to the age, sex, weight, condition, degree of disease, drug form, administration route and period of the subject to be administered, but generally as about 0.05 to 500 mg/kg, preferably, about 0.1 to 250 mg/kg may be administered 1 to 3 times a day.

It is apparent to those skilled in the art that the formulation method, dosage, route of administration, components, etc. of the pharmaceutical composition of the present invention may be appropriately selected from conventional techniques known in the art.

The pharmaceutical composition of the present invention can be used for the prevention and treatment of viral infectious diseases. The antiviral pharmaceutical composition of the present invention may include other pharmaceutically active ingredients in addition to the peptide of the present invention as an active ingredient or may be used in combination with a pharmaceutical composition including other active ingredients.

As used herein, the term “composition for preventing or treating infection” is a biological preparation comprising an antigen that gives immunity to a living body, and a vaccine composition or pharmaceutical composition that is an immunogen or antigenic substance that generates immunity in a living body by injecting or oral administration to a person or animal for the prevention of infection may be included.

As used herein, the term “prevention” refers to all actions in which suppress or delay the corona virus infection by administering a transformant expressing the peptide or a composition comprising the transformant and/or the peptide of the present invention as an active ingredient.

As used herein, the term “treatment” refers to all actions in which the symptoms of coronavirus infection are improved or beneficial by administering a transformant expressing the peptide or a composition comprising the transformant and/or the peptide of the present invention as an active ingredient.

“Immunogen” or “antigenic substance” may be any one selected from the group consisting of peptides, polypeptides, strains expressing the polypeptides, proteins, oligonucleotides, polynucleotides, recombinant bacteria and recombinant viruses. As a specific example, the antigenic material may be in the form of an inactivated whole or partial cell preparation, or in the form of an antigenic molecule obtained by conventional protein purification, genetic engineering technique, or chemical synthesis.

The pharmaceutical composition for preventing or treating coronavirus infection comprising the protein extract of the transformant described above or the recombinant coronavirus peptide isolated from the transformant of the present invention can be formulated in various forms, such as oral formulations like powders, granules, tablets, capsules, suspensions, emulsions, syrups, aerosols, and injection formulation of sterile injection solutions, according to conventional methods for each purpose of use. prepared according to a conventional method for each purpose of use, and it may be administered orally or via various routes including intravenous, intraperitoneal, subcutaneous, rectal, topical, and the like.

The pharmaceutical composition may further include a carrier, excipient or diluent, and the like, and examples of suitable carriers, excipients or diluents that may be included include lactose, dextrose, sucrose, sorbitol, mannitol, xylitol, erythritol, maltitol, Starch, gum acacia, alginate, gelatin, calcium phosphate, calcium silicate, cellulose, methyl cellulose, amorphous cellulose, polyvinyl pyrrolidone, water, methyl-hydroxybenzoate, propyl-hydroxybenzoate, talc, magnesium stearate and mineral oil. and the like. In addition, the pharmaceutical composition of the present invention may further include a filler, an anti-agglomeration agent, a lubricant, a wetting agent, a fragrance, an emulsifier, a preservative, and the like.

The active ingredient of the present invention is administered in a pharmaceutically effective amount. In the present invention, “pharmaceutically effective amount” means an amount sufficient to treat a disease with a reasonable benefit/risk ratio applicable to medical treatment, and the effective dose level is determined by the type, and severity of disease in the patient, activity of the drug, sensitivity to the drug, time of administration, route of administration and excretion rate, duration of treatment, factors including concomitant drugs, and other factors well known in the medical field. The pharmaceutical composition of the present invention may be administered as an individual therapeutic agent or may be administered in combination with other therapeutic agents, may be administered sequentially or simultaneously with conventional therapeutic agents, and may be administered single or multiple. In consideration of all the above factors, it is important to administer an amount that can obtain the maximum effect with a minimum amount without side effects, which can be easily determined by those skilled in the art.

The effective amount of the active ingredient in the pharmaceutical composition of the present invention may vary depending on the age, sex, and weight of the patient, and generally 0.001 to 5,000 mg, preferably 0.1 to 3,000 mg per kg of body weight, is administered daily or every other day or 1 It can be administered in divided doses 1 to 3 times a day. However, since it may increase or decrease depending on the route of administration, the severity of the disease, sex, weight, age, etc., the dosage is not intended to limit the scope of the present invention in any way.

The pharmaceutical composition of the present invention may be administered to a subject through various routes. Any mode of administration can be envisaged, for example, by oral, rectal or intravenous, intramuscular, subcutaneous, intrauterine dural or intracerebroventricular injection.

In the present invention, “administration” means providing a predetermined substance to a patient by any suitable method, and the administration route of the pharmaceutical composition of the present invention is oral or parenteral through all general routes as long as it can reach the target tissue. It can be administered orally. In addition, the composition of the present invention may be administered using any device capable of delivering an active ingredient to a target cell.

In the present invention, “subject” is not particularly limited, but includes, for example, humans, monkeys, cattle, horses, sheep, pigs, chickens, turkeys, quails, cats, dogs, mice, rats, rabbits or guinea pigs. and preferably a mammal, more preferably a human.

Hereinafter, the present invention will be described.

In the present invention, the present inventors newly discovered a direct interaction between the N protein and the spike (S) protein in MERS-CoV-infected cells through coimmunoprecipitation and proteomics analysis. The synthesized peptide corresponding to the C-terminal domain of the S protein (Spike CD) inhibited the interaction of the N protein with the spike (S) protein in vitro. In addition, cell penetration of the spike CD peptide inhibited viral plaque formation in MERS-CoV-infected cells.

In addition, it was confirmed that the cellular penetration of SARS-CoV-2 spike CD peptide inhibited SARS-CoV-2 virus production by quantitative real-time RT-PCR.

In addition, a direct interaction between the N protein and the spike (S) protein was newly discovered in HCoV-OC43-infected cells. Intracellular penetration of the synthesized peptide corresponding to the C-terminal domain of the S protein (Spike CD) was confirmed, by confocal microscopy that the production of the N protein and the spike (S) protein of HCoV-OC43 was reduced in HCoV-OC43-infected cells and inhibition of HCoV-OC43 virus production was confirmed by plaque formation experiment. Therefore, we propose that understanding and targeting the interaction of the S protein with the N protein will be helpful for future treatments against coronaviruses including MERS-CoV, SARS-CoV-2 and HCoV-OC43.

Hereinafter, the present invention will be described in detail.

### Association of MERS-CoV S Protein and N Protein

To investigate S protein- or M protein-interacting protein(s) in MERS-CoV-infected cells, we performed immunoprecipitation with the monoclonal antibody (492-1G10E4E2 clone) against the MERS-CoV S protein (anti-S mAb) or monoclonal antibody (M158-2D6F11 clone) against the MERS-CoV M protein (anti-M mAb) in the MERS-CoV-infected Vero cell lysates and analyzed co-immunoprecipitated proteins by proteomics approach. When the expression of S, M and N protein in the MERS-CoV-infected cells was monitored for 72 h by Western blotting, N protein expression increased most rapidly and robustly than others after MERS-CoV infection (FIGS. 1 to 4).

As shown in FIG. 5 (arrowhead), S protein-interacting protein with a molecular weight of ˜45 kDa was stained in the gel obtained by SDS-PAGE. The interacting protein was fragmented in gel with trypsin and the peptide fragments were analyzed by ESI-TOF MS/MS. The result revealed 19 matched peptide fragments with 57.14% sequence coverage of the total amino acids of MERS-CoV N protein (FIG. 9). This result verifies that S protein majorly interacts with N protein in the MERS-CoV-infected cells.

To confirm the interaction of S protein and N protein, immunoprecipitation and Western blotting analysis were performed using MERS-CoV-infected cell lysates. N protein was detected in the immunocomplex obtained by anti-S mAb, which agrees with mass spectroscopy analysis (FIG. 6).

However, M protein was not found in the immunocomplexes. As shown in FIG. 7, prominent M protein-interacting proteins were not detected in SDS-PAGE. However, the analysis of immunocomplex obtained by anti-M mAb showed interaction of N protein with M protein in MERS-CoV-infected cells (FIG. 8) as previously reported in SARS-CoV ((Z. J. Miknis, E. F. Donaldson, T. C. Umland, R. A. Rimmer, R. S. Baric, L. W. Schultz, J. Virol. 83, 3007-3018 (2009)) and MHV-infected cells (K. R. Hurst, L. Kuo, C. A. Koetzner, R. Ye, B. Hsue, P. S. Masters, J. Virol. 13285-13297 (2005)).

In addition, small amount of S protein was detected in the immunocomplex of anti-M mAb Immunoprecipitation with anti-N mAb and Western blotting with either anti-S mAb or anti-N Ab were performed to further confirm the interaction of N protein with S protein and M protein in a reciprocal manner.

Interaction of Spike CD of MERS-CoV with N Protein

We next checked the specificity of interaction between S protein of MERS-CoV with N protein in detail.

Because Spike CD can possibly bind with N protein in cells and in the assembled virus, we synthesized four kinds of peptides covering full Spike CD (Spike CD-Full), front region of Spike CD (Spike-F), middle region of Spike CD (Spike-M), back region of Spike CD (Spike-B), to identify the interaction (FIG. 10).

Each biotinylated-Spike CD peptide (Spike CD-Full-Biotin, Spike-F-Biotin, Spike-M-Biotin, or Spike-B-Biotin) was mixed with MERS-CoV-infected cell lysates and the binding of the Spike CD peptides with N protein was confirmed by immunocomplex analysis using Streptavidin-beads.

FIG. 11 shows that Spike CD peptides interacted with N protein and the peptide of transmembrane domain proximal region (Spike CD-F) can tightly bind with N protein.

To further investigate the key interaction region between MERS-CoV spike CD and N protein, the interaction of N protein with total Spike-CD-biotin in MERS-CoV-infected cell lysates was analyzed after pretreatment with Spike CD-F, Spike CD-M or Spike CD-B.

As shown in FIG. 12, all these peptides can inhibit 30-40% of the interaction between Spike CD-Full and N protein with the highest activity in the presence of Spike-CD-F. This apparent small effect can be understood in the context that N protein in the cell lysates is already bound with interacting proteins including virus-derived S protein. Taken together, we can conclude that Spike CD-F covers the most important region interacting with N protein, but Spike-CD Full is the best peptide to study the interaction.

### Cell Permeation of the MERS-CoV Spike CD Peptide Inhibits MERS-CoV Production.

To determine whether the peptide spike-CD-Full (full) of MERS-CoV can inhibit the interaction between the S protein and the N protein in the cell, thereby inhibiting the production of MERS-CoV in the cell, a cell-penetrating peptide strategy was performed.

We synthesized nine D-arginine-conjugated whole spike CD-peptides (R-Spike CD) and a derivative with biotin at the C-terminus (R-Spike CD-Biotin). R-spike CD-biotin was treated in Vero cells, and then peptide uptake was monitored by confocal microscopy after 30 min. Confocal images showed that the fluorescence intensity increased in the cytoplasm in a dose-dependent manner as expected (FIG. 13).

To investigate the cell-penetrating effect of Spike CD peptide on the expression of S, M and N proteins in MERS-CoV-infected cells, Vero cells were infected with MERS-CoV (0.1 MOI) in the presence or absence of R-Spike CD. According to western blotting data, the expression of S, M and N proteins was significantly increased at 48 hours after infection, and protein expression was decreased by R-spike CD treatment (FIG. 14). In contrast, confocal images show that the expression of S protein was greatly reduced by R-Spike CD, presumably due to the high sensitivity of this assay (FIG. 15).

To investigate the cell-penetrating effect of Spike CD peptide on the production of MERS-CoV in cells, Vero cells were infected with MERS-CoV in the presence of R-Spike CD. FIGS. 16 and 17 show that plaque formation was reduced when cells were treated with R-Spike CD in a dose-dependent manner. However, no effect was observed with the control cell penetrating peptide (R-CP-1). These results confirmed that inhibition of the interaction between Spike CD and N protein by the cell-permeable Spike CD peptide reduced MERS-CoV amplification.

### Cell Penetration of Spike CD Peptide of SARS-CoV-2 Inhibits SARS-CoV-2 Production

To determine if the spike-CD peptide of SARS-CoV-2 can inhibit the interaction between the S protein and the N protein of SARS-CoV-2 in the cell, thereby inhibiting the production of SARS-CoV-2 in the cell, we used a cell penetrating peptide strategy.

We synthesized nine D-arginine-conjugated spike CD-COVID-19 peptides (R-Spike CD-COVID-19). To investigate the cell-penetrating effect of the spike CD-COVID-19 peptide on the production of SARS-CoV-2 in cells, Vero cells were infected with SARS-CoV-2 and the CD-COVID-19 peptide was administered 6 hours later. FIG. 18 shows that SARS-CoV-2 production was reduced by 70% when cells were treated with cell-permeable Spike CD-COVID-19 (R-Spike CD-COVID-19). However, it was observed that the effect was as low as 40% by the control cell-penetrating peptide (R-CP-1) and the cell-penetrating MERS-CoV spike CD peptide (R-Spike-CD(MERS)). These results confirmed that inhibition of the interaction between Spike CD and N protein by the cell-permeable Spike CD-COVID-19 peptide reduced SARS-CoV-2 amplification.

### Cell Penetration of HCoV-OC43 Spike CD Peptide Inhibits HCoV-OC43 Production.

To confirm the interaction of the S protein with the N protein of HCoV-OC43, immunoprecipitation and western blotting analysis were performed using HCoV-OC43-infected cell lysates. N protein was detected in immune complexes obtained by anti-S Ab (FIG. 19).

To determine whether the spike-CD peptide of HCoV-OC43 (Spike CD-OC43) can inhibit the interaction between the S protein and the N protein of HCoV-OC43 in the cell, thereby inhibiting the production of HCoV-OC43 in the cell, a cell penetrating peptide strategy was used.

We synthesized nine D-arginine-conjugated spike CD-OC43 peptides (R-Spike CD-OC43). To investigate the cell-penetrating effect of the spike CD-OC43 peptide on the production of HCoV-OC43 in cells, Vero cells were infected with HCoV-OC43 and the CD-OC43 peptide was administered 6 hours later. FIGS. 20 to 21 and 22 show that the production of S protein and N protein of a cell penetrating HCoV-OC43 was reduced when cells were treated with the cell-permeable spike CD-OC43. However, no effect was observed with the control peptide. FIG. 23 also shows that HCoV-OC43 production was reduced by 20% when cells were treated with the cell-permeable spike CD-OC43. These results confirmed that inhibition of the interaction between the spike CD and N protein by the cell-permeable spike CD-OC43 peptide reduced HCoV-OC43 amplification.

### Advantageous Effects

As can be seen from the present invention, the peptides of the present invention based on the understanding and targeting of the interaction between the coronavirus S protein and the N protein. have an effect that may be helpful in the treatment of coronaviruses comprising MERS-CoV and SARS-CoV-2 and SARS-CoV and HCoV-OC43.

### DESCRIPTION OF DRAWINGS

FIGS. 1 to 4 are diagrams showing the expression of S, M and N proteins in MERS-CoV-infected cells at 72 h. Cell lysates were prepared from Vero cells and MERS-CoV-infected Vero cells, and 50 μg (FIGS. 1, 2, 4) and 25 μg (FIG. 3) of protein containing cell lysates were separated by 4-12% gradient SDS-PAGE and analyzed by Western blotting with the indicated antibodies. The exposure times for signal detection were 60 s (FIGS. 1, 2, 4) and 5 s (FIG. 3), respectively,

FIGS. 5 to 8 are diagrams showing the interaction of MERS-CoV S protein and M protein with MERS-CoV N protein, (FIGS. 5 and 7) with the identification of S protein (FIG. 5) and M protein (FIG. 7) binding proteins, cell lysates were prepared from Vero cells and MERS-CoV-infected Vero cells. Cell lysates (150 μg protein) were immunoprecipitated with anti-S mAb (FIG. 5) or anti-M mAb (FIG. 7), then separated by 4-12% gradient SDS-PAGE and was stained with Coomassie Brilliant Blue G-250. The indicated (arrowhead) protein bands on the gel were digested with trypsin, and the treated peptides were analyzed by ESI-TOF MS/MS. HC, heavy chain. LC, light chain. (FIGS. 6, and 8) To show the association of S protein and M protein with N protein, cell lysates were prepared from Vero cells and MERS-CoV-infected Vero cells. Lysates were immunoprecipitated with anti-S mAb (FIG. 6) or anti-M mAb (FIG. 8) and immune complexes were subjected to Western blotting with the described antibodies. The loading of immunoprecipitated samples for N protein (anti-N Ab) analysis was half the amount for analysis of others (anti-S mAb, anti-M mAb) (FIGS. 6, and 8). The exposure times for signal detection were 120 s (anti-S mAb, anti-M mAb) and 5 s (anti-N Ab), respectively,

FIG. 9 is a diagram showing the identification of the MERS-CoV S protein-binding protein. The protein band co-immunoprecipitated with the MERS-CoV S protein was treated on a gel with trypsin, and the peptide was analyzed by ESI-TOF MS/MS. MS/MS analysis of the mass peak (arrow) obtained in the ˜45 kDa band shows the peptide spectrum of the MERS-CoV N protein,

FIGS. 10 to 12 are diagrams showing the interaction between the cytoplasmic domain of the MERS-CoV S protein and the MERS-CoV N protein (FIG. 10) shows a schematic diagram and the sequence of the cytoplasmic domain of the S protein, RBD, receptor binding domain; FP, fusion peptide; HR1 and HR2, heptad repeat regions 1 and 2; TM, transmembrane; CD, C-terminal domain; Spike CD, C-terminal domain of MERS-CoV S protein; Spike CD-full, MERS-CoV Spike CD-F, Spike CD-M and Spike CD-B represent synthetic peptide sequences. (FIG. 11) Representing the immunoprecipitation assay, cell lysates were prepared from Vero cells and MERS-CoV-infected Vero cells. Western blot analysis using anti-N Ab was performed on immune complexes obtained from each biotinylated synthetic peptide sequence. The right column shows the relative band intensities of the N protein. (FIG. 12) Showing competition of Spike CD peptide for the interaction of MERS-CoV Spike CD with MERS-CoV N protein, cell lysates were prepared from MERS-CoV-infected Vero cells. Lysates were incubated for 2 h at 37° C. with each of the Spike CD-F, Spike CD-M and Spike CD-B peptides and then with biotinylated Spike CD-Full-Biotin at 37° C. incubated for 2 hours. Western blot analysis using an anti-N protein antibody was performed on the immune complexes obtained by streptavidin-beads. The right column shows the relative band intensities of the N protein,

FIG. 13 is a diagram showing the location of R-Spike CD in Vero cells. Vero cells were cultured for 24 hours and then incubated with R-Spike CD-biotin peptide at 37° C. for 30 minutes in a 5% CO₂ incubator. Samples were fixed with 4% paraformaldehyde and permeabilized with 0.1% Triton X-100. Cell-permeated R-spike CD-biotin peptide was detected with Alexa flour-488-conjugated streptavidin (green) by Carl Zeiss LSM710. Nuclei were stained with Hoechst 33258 (blue). (It's black and white, but you can't see the blue green . . . .) Scale bar, 10 μm. R-spike CD, a peptide corresponding to the C-terminal domain of the MERS-CoV S protein with nine D-arginines at the N-terminus; R-Spike CD-Biotin, Biotinylated R-Spike CD Peptide,

FIGS. 14 to 17 are diagrams showing the effect of R-Spike CD on MERS-CoV production, (FIGS. 14 and 15) showing a decrease in MERS-CoV protein production by R-Spike CD, (FIG. 14) Cells lysates were prepared at the indicated time points from Vero cells infected with MERS-CoV (with or without R-Spike CD). Cell lysates were analyzed by Western blotting using the indicated antibodies. (FIG. 15) Vero cells were infected with MERS-CoV (with or without R-Spike CD peptide) in serum-free medium. Cells were incubated for 48 h, then stained with anti-S mAb followed by Alexa Flour 488-conjugated goat anti-mouse IgG antibody and analyzed by confocal microscopy. Scale bar, 20 μm. (FIGS. 16 and 17) To show inhibition of MERS-CoV plaque formation by R-Spike CD, MERS-CoV was mixed with 2-fold serially diluted R-Spike CD and R-CP-1. The MERS-CoV virus-peptide mixture was treated to Vero cells in a 5% CO₂ incubator at 37° C. After incubation for 1 hour, the medium was removed and then supplemented with DMEM/F12 containing 0.6% Oxoid agar. After culturing for 4 days, plaque formation was confirmed by staining with crystal violet. (FIG. 16) Representative photographs showing plaque formation. (FIG. 17) shows the quantification of plaques obtained by MERS-CoV infection after treatment with each peptide from 0 μM to 100 μM, where the number of plaques obtained from control plates treated with only MERS-CoV virus was 100%. R-spike CD, a peptide corresponding to the C-terminal domain of the S protein with 9 D-arginines at the N-terminus; R-CP-1, nine D-arginine-conjugated control peptides.

FIG. 18 is a diagram showing the effect of R-Spike CD-COVID-19 on SARS-CoV-2 production, showing inhibition of SARS-CoV-2 production by cell-permeable Spike CD-COVID-19 peptide (R-Spike CD-COVID-19), SARS-CoV-2 (0.1 MOI) was incubated at 37° C. with 5% CO₂ incubator. treated with Vero cells. After incubation for 1 hour, the medium was removed and then supplemented with DMEM containing 2% FBS. After incubation for 6 h, the cell-permeable Spike CD-COVID-19 peptide (R-Spike CD-COVID-19), the control cell-penetrating peptide (R-CP-1) and the cell-permeable MERS-CoV spike CD peptide (R-Spike CD-COVID-19)-Spike CD (MERS), each 5 μM were treated respectively. 24 hours after SARS-CoV-2 infection, viral RNA was isolated from the cell culture medium, and cDNA was prepared. The produced virus was quantified by performing real-time RCR using primers for SARS-CoV-2 RNA-dependent RNA polymerase (RdRP). R-spike CD-COVID-19, a peptide corresponding to the C-terminal domain of SARS-CoV-2 S protein with 9 D-arginines at the N-terminus; R-spike CD, a peptide corresponding to the C-terminal domain of the MERS-CoV S protein with nine D-arginines at the N-terminus; R-CP-1, nine D-arginine-conjugated control peptides,

FIG. 19 is a diagram showing the expression of S and N proteins and the interaction of HCoV-OC43 S protein with HCoV-OC43 N protein in HCoV-OC43-infected cells at 72 hours, (A) Cell lysates prepared from OC43-infected Vero cells and cell lysates were separated by 4-12% gradient SDS-PAGE and analyzed by Western blotting with the indicated antibodies. (B) The association of S protein with N protein. Cell lysates were prepared from Vero cells and HCoV-OC43-infected Vero cells. Lysates were immunoprecipitated with anti-S Ab and immune complexes were subjected to western blotting with anti-N mAbs with the described antibodies. HC, heavy chain. LC, light chain. S, S protein. N, N protein,

FIG. 20 is a diagram showing the effect of R-Spike CD-OC43 on the production of HCoV-OC43 S and N proteins, showing a decrease in HCoV-OC43 protein production by R-Spike CD-OC43, and the cell lysate was prepared from Vero cells infected with HCoV-OC43 (presence or absence of R-Spike CD-OC43) 48 hours later. Cell lysates were analyzed by Western blotting using the indicated antibodies.

FIGS. 21 to 22 are diagrams showing the effects of R-Spike CD-OC43 on the production of HCoV-OC43 S and N proteins. Vero cells were infected with HCoV-OC43 (with or without R-Spike CD-OC43). After incubating the cells for 48 h (FIG. 21), S protein was analyzed by confocal microscopy after staining with anti-S Ab and then Alexa Flour 488-conjugated goat anti-rabbit IgG antibody. (FIG. 22) N protein was analyzed by confocal microscopy after staining with anti-N mAb followed by Alexa Flour 488-conjugated rabbit anti-mouse IgG antibody. Scale bar, 20 μm,

FIG. 23 is a diagram showing the effect of R-Spike CD-OC43 on HCoV-OC43 virus production, showing the inhibition of HCoV-OC43 production by cell-permeable spike CD-OC43 peptide (R-Spike CD-OC43); HCoV-OC43 (0.1 MOI) was treated with Vero cells in a 5% CO₂ incubator at 37° C. After incubation for 1 hour, the medium was removed and then supplemented with DMEM containing 2% FBS. After incubation for 6 hours, cell penetrating spike CD-OC43 peptide (R-Spike-CD-OC43) and control spike CD-OC43 peptide (Spike-CD-OC43) (2 μM each) were each treated. After 48 hours of HCoV-OC43 infection, the virus produced in the cell culture was quantified by a plaque formation test.

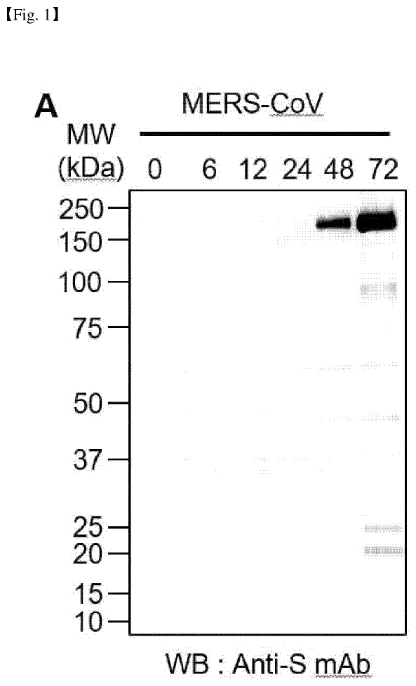

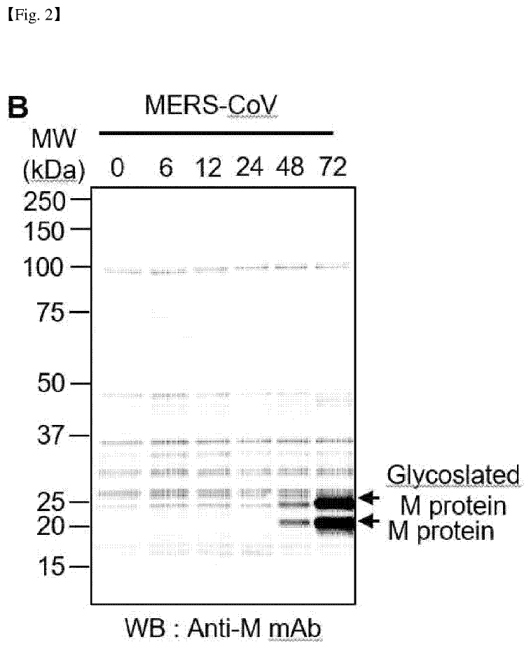

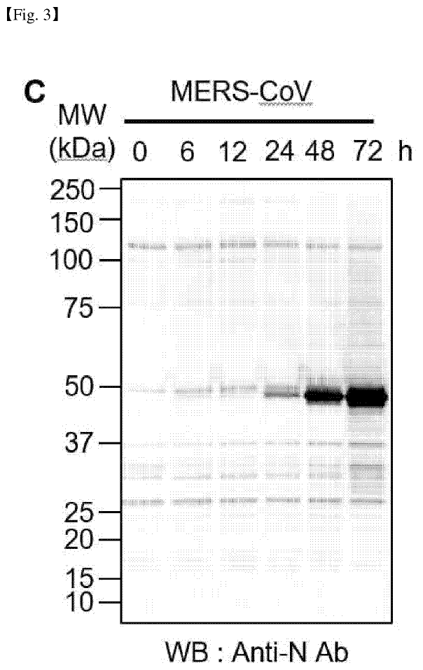

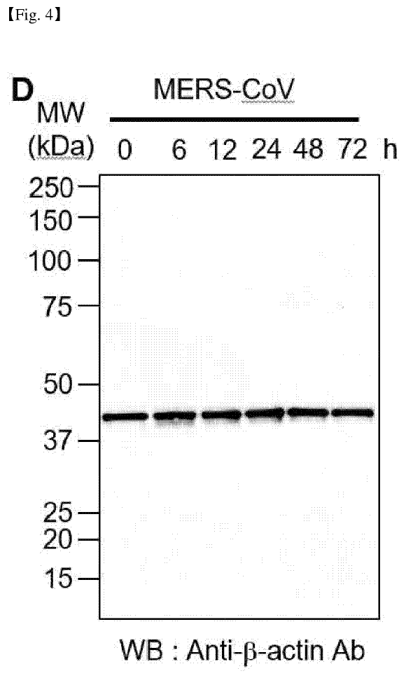

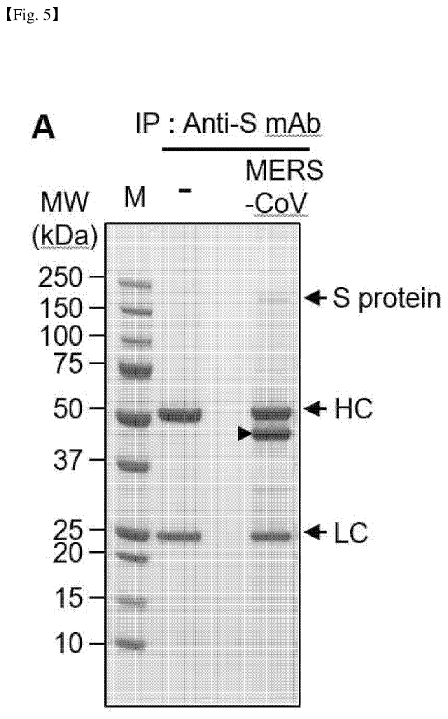

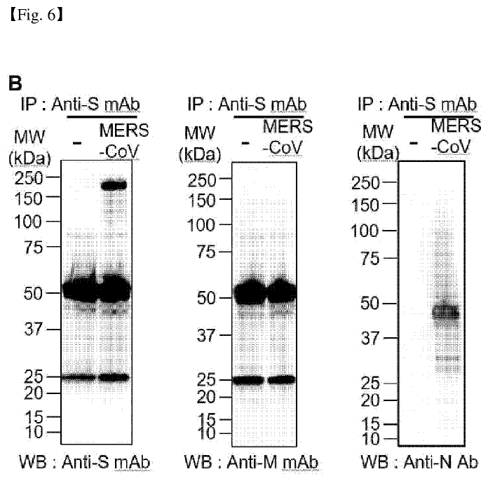

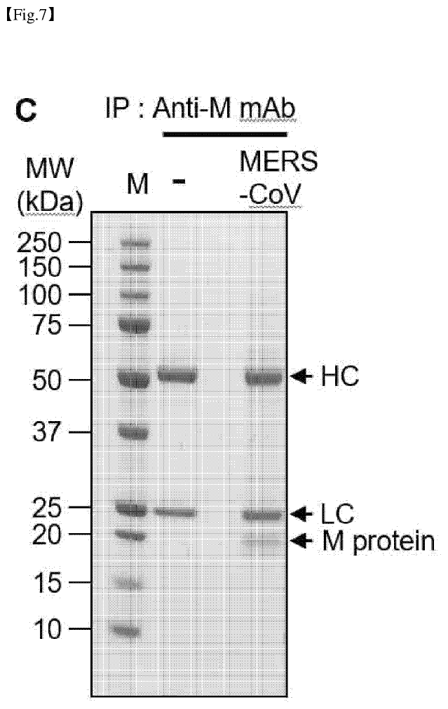

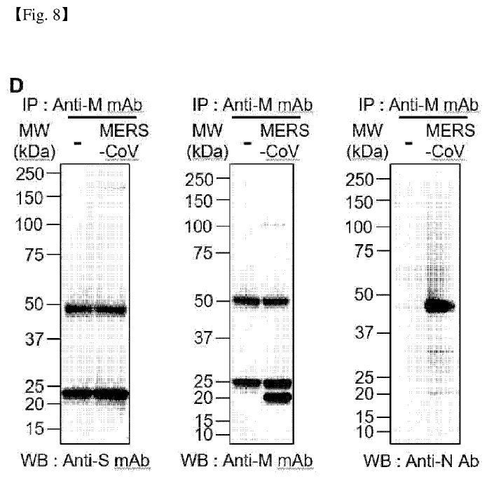

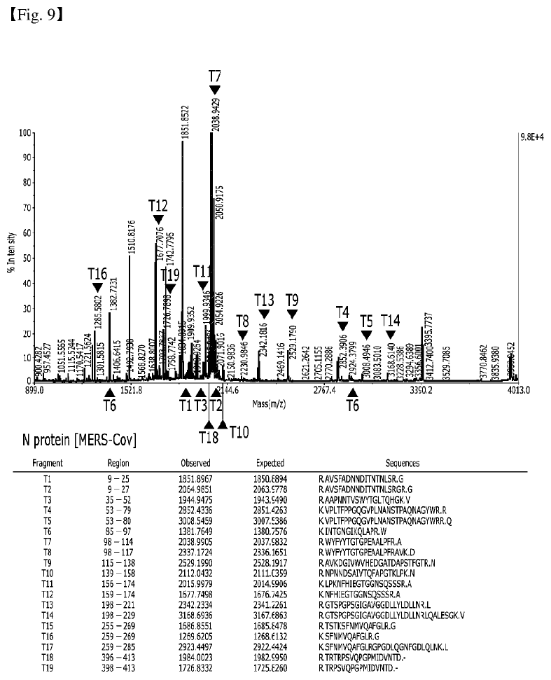

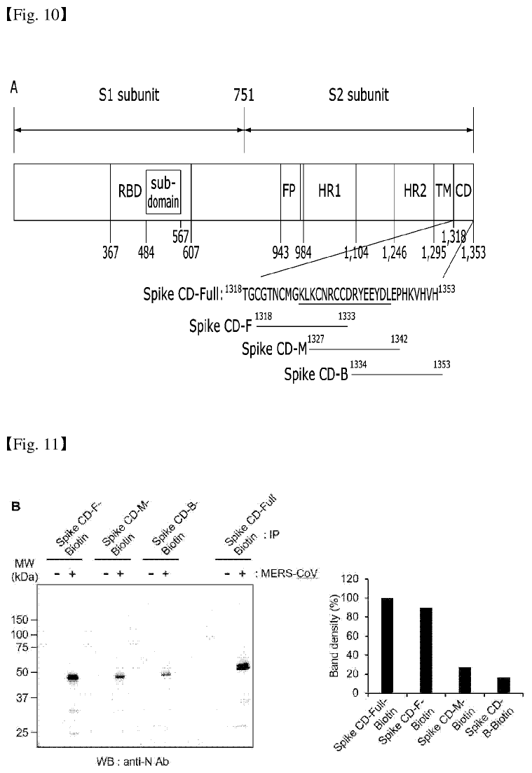

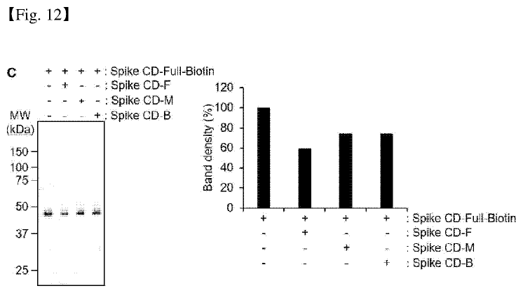

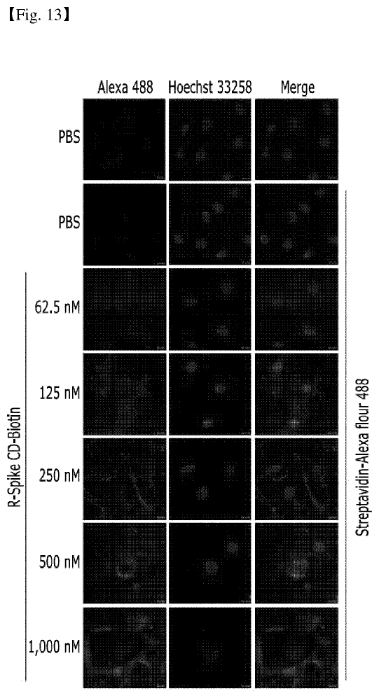

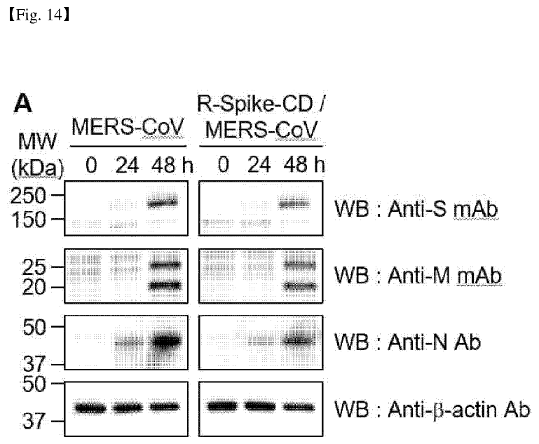

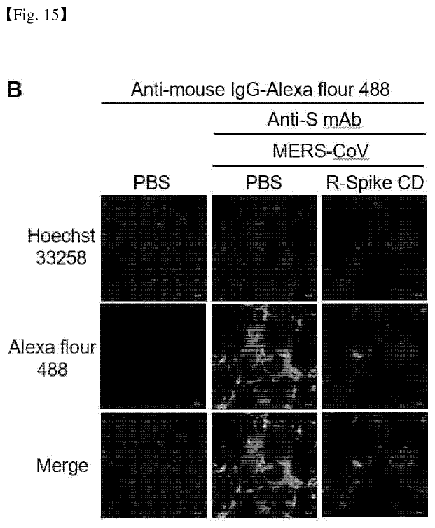

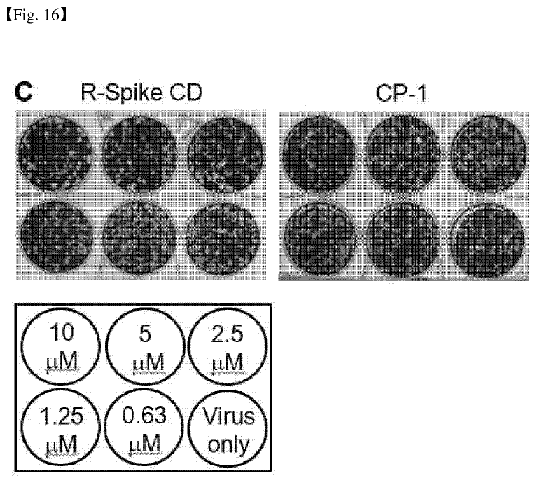

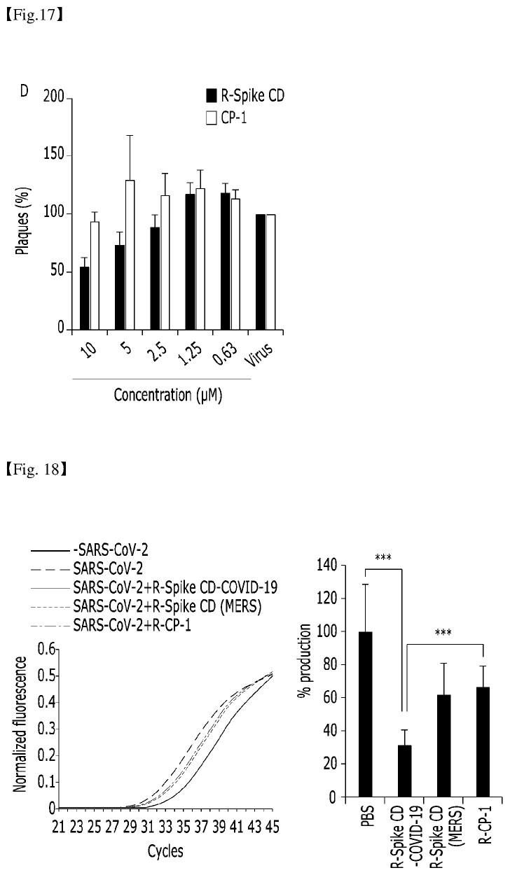

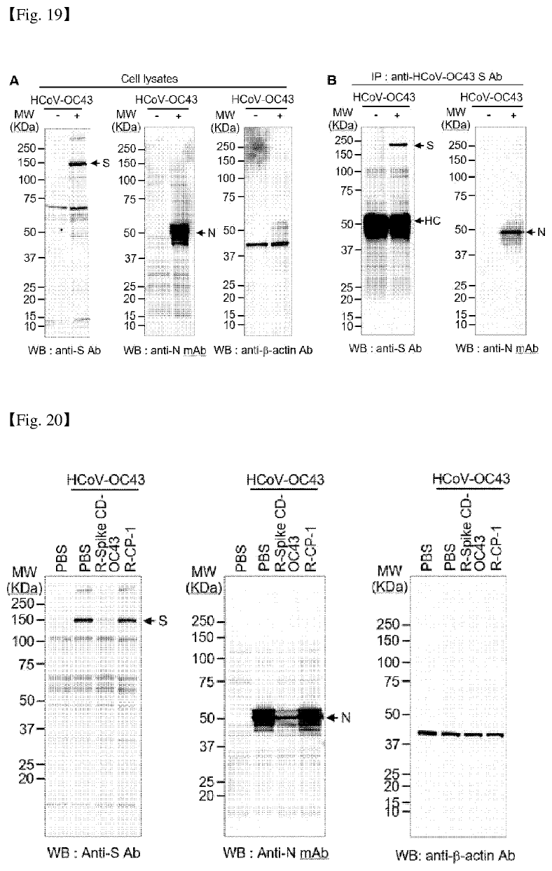

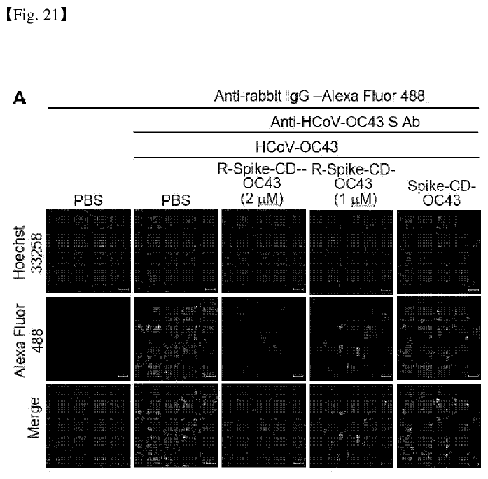

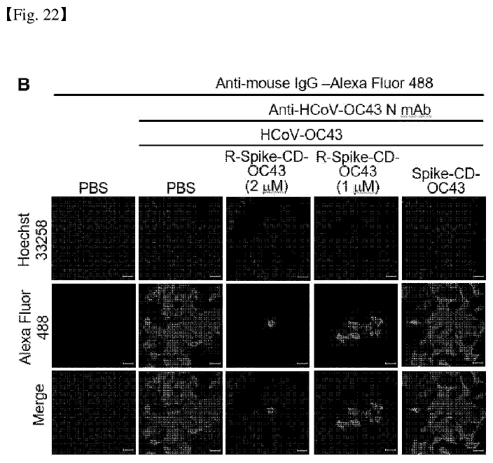

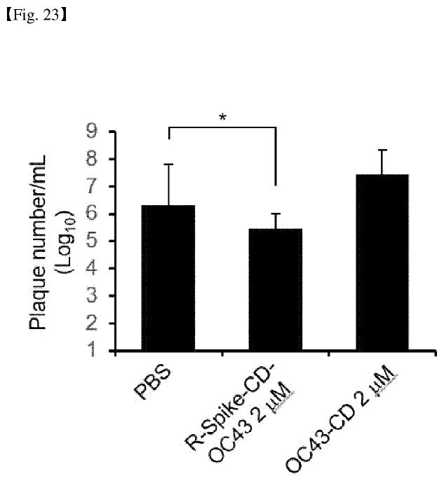

### MODE FOR INVENTION

Hereinafter, the present invention will be described in more detail by the following examples. However, the following examples are described with the intention of illustrating the present invention, and the scope of the present invention is not to be construed as being limited by the following examples.

### Example 1: Cell Lines and Viruses

Vero cells, Vero E6 cells and Calu-3 cells were purchased from the Korean Cell Line Bank (Seoul, Korea). Cells was cultured in Dulbecco's Modified Eagle's Medium (DMEM, Thermo Fisher Scientific, MA, USA) containing 10% fetal bovine serum (FBS, Thermo Fisher Scientific), 25 mM HEPES, 100 U/ml penicillin and 100 μg/ml streptomycin. Cells were incubated at 37° C. in atmospheric conditions of 95% air and 5% CO₂. MERS-CoV/KOR/KNIH/002_05_2015 and SARS-CoV-2 (NCCP No. 43326) were obtained from the Korea Disease Control and Prevention Agency. Virus preparation and cell culture procedures were performed under biosafety level 3 (BSL-3) conditions. HCoV-OC43 (KBPV-VR-8) was obtained from the Korea Bank for Pathogenic Viruses (Korea University). HCoV-OC43 preparation and cell culture procedures were performed under biosafety level 2 (BSL-2) conditions.

### Example 2: Peptide Synthesis

The spike CD of MERS-CoV was analyzed from the MERS-CoV S protein sequence (MERS-CoV/KOR/KNIH/002_05_2015 (GI: 829021049)), and the following peptides were designed to investigate the interaction between S protein and N protein:

Spike CD-F,
(SEQ ID NO: 1)
¹³¹⁸TGCGTNCMGKLKCNRC¹³³³.
Spike CD-M,
(SEQ ID NO: 2)
¹³²⁷KLKCNRCCDRYEEYDL¹³⁴³.
Spike CD-B,
(SEQ ID NO: 3)
¹³³⁶DRYEEYDLEPHKVHVH¹³⁵³.
Spike CD-Full,
(SEQ ID NO: 4)
¹³¹⁸TGCGTNCMGKLKCNRCCDRYEEYDLEPHKVHVH¹³⁵³.

All peptides were synthesized by an automatic peptide synthesizer (Anygen Co., LTD. Gwangju). In order to penetrate the peptides into cells, each peptide is conjugated with nine D-arginines on the N-terminus (R-Spike CD) and/or with biotin on the C-terminus (R-Spike CD-Biotin, Spike CD-Full-Biotin, Spike CD-F-Biotin, Spike CD-M-Biotin, Spike CD-B-Biotin)) and 9 D-Arginine-conjugated control peptides R-CP-1 (NH2-d-RRRRRRRRRR-AQARRKNYGQLDIFP-COOH; (SEQ ID NO: 5)) was used as a control cell penetrating peptide (D. Raina, et al. Cancer Res. 69, 5133-5141 (2009)).

The spike CD of SARS-CoV-2, a coronavirus infection-19 coronavirus, was synthesized from the SARS-CoV-2 S protein sequence (QHD43416) and the spike CD from SARS-CoV, a severe acute respiratory syndrome coronavirus, with the SARS-CoV S protein sequence (NP_828851.1), and in order to penetrate the peptide into cells, the following peptide conjugated with the spike CD peptide of SARS-CoV-2 with 9 D-arginines on the N-terminus (R-spike CD-COVID-19) was designed: SARS-CoV-2 is the name of the virus that causes COVID-19 (disease name), and when naming the peptide, SARS-CoV-2 is too long and named COVID-19.

Spike CD-COVID-19,
(SEQ ID NO: 6)
¹²³⁴LCCMTSCCSCLKGCCSCGSCCKFDEDDSEPVLKGVKLHYT¹²⁷³
Spike CD-SARS-CoV
(SEQ ID NO: 7)
¹²¹⁶LCCMTSCCSCLKGACSCGSCCKFDEDDSEPVLKGVKLHYT¹²⁵⁵

Spike CD-SARS-CoV has one different amino acid sequence from Spike CD-COVID-19, so a separate experiment was not performed by synthesizing Spike CD-SARS-CoV.
The spike CD of human coronavirus OC43 (HCoV-OC43) was analyzed from the HCoV-OC43 S protein sequence (YP_009555241.1), and to penetrate the peptide into cells, the following peptide conjugated with the spike CD peptide of HCoV-OC43 with 9 D-arginines (R-spike CD-OC43) was designed.

Spike CD-OC43’
(SEQ ID NO: 8)
¹³²⁰CCTGCGTSCFKKCGGCCDDYTGYQELVIKTSHDD¹³⁵³

### Example 3: Antibody

A monoclonal antibody (492-1G10E4E2 clone) against MERS-CoV S protein (anti-S mAb) (B. K. Park, S. Maharjan, S. I. Lee, J. Kim,J-Y. Bae, M.-S. Park, H.-J. Kwon, BMB Rep 52, 397-402 (2019)) and a monoclonal antibod(M158-2D6F11 clone) against MERS-CoV M protein (anti-M mAb) (B. K. Park, S. I. Lee, J.-Y. Bae, M.-S. Park, Y. Lee, H.-J. Kwon, Int J Pept Res Ther, 1-8 (2018)) was prepared, as described in D. Kim, S. Kwon, J. W. Rhee, K. D. Kim, Y.-E. Kim, C.-S. Park, M. J. Choi, J.-G. Suh, D.-S. Kim, Y. Lee, BMC Immunol. 12, 29 (2011), each peptide epitope formulated into a CpG-DNA-liposome complex was used to prepare from hybridoma cells established after immunization of BALB/c mice.

Spike-492 (⁴⁹²TKPLKYSYINKCSRLLSDDRTEVPQ⁵¹⁶; (SEQ ID NO: 9)) and MERS-M158 (¹⁵⁸CDYDRLPNEVTVAKPNVLIALKMVK¹⁸²; (SEQ ID NO: 10)) were used as B cell epitope sequences for the S protein (Spike glycoprotein universal sequence (GI: 510785803)) and M protein of MERS-CoV, respectively. Rabbit anti-MERS N protein antibody (anti-N Ab) was purchased from Sino Biological (Cat. No. 40068-RP02, Vienna, Austria) and anti-β-actin antibody was purchased from Sigma-Aldrich (St. Louis, Mo., USA).

Mouse anti-HCoV-OC43 N protein antibody (anti-N mAb) was purchased from LSBio (Cat No. LS-C79764, Seattle, USA), and rabbit anti-HCoV-OC43 S protein antibody (anti-S Ab) was purchased from LSBio (Cat No. LS-C371066).

### Example 4: MERS-CoV Infection and Co-Immunoprecipitation Method

Vero cells were cultured for 12 hours at a density of 6×10⁵ cells/10 cm dish. MERS-CoV (0.1 MOI) was inoculated into Vero cells in serum-free medium, and then incubated at 37° C. in a 5% CO₂ incubator for 1 hour. After incubation, the supernatant was removed and each dish was replenished with DMEM medium containing 25 mM HEPES, 100 U/ml penicillin and 100 μg/ml streptomycin. 3 days post infection, MERS-CoV-infected Vero cells were lysed in cell lysis buffer (10 mM HEPES, 150 mM NaCl, 5 mM EDTA, 100 mM NaF, 2 mM Na₃VO₄, protease inhibitor cocktail and 1% NP-40) at 4° C. for 30 min. Cell lysates were centrifuged to remove cell debris, and cell lysates were incubated with anti-S mAb or anti-M mAb at 4° C. for 3 hours.

Protein A beads (CaptivAtm PriMAB 52% (v/v) slurry, REPLIGEN, Waltham, Mass., USA) were added, followed by centrifugation to collect immune complexes Immune complexes were separated by 4-12% gradient SDS-PAGE (Bottom 4-12% Bis-Tris Plus gel, Thermo Fisher Scientific) and stained with Coomassie Brilliant Blue G-250.

### Example 5: MERS-CoV S Protein Binding Protein Analysis

After immunoprecipitation with anti-S mAb in MERS-CoV-infected cell lysates, the immune complexes were separated by 4-12% gradient SDS-PAGE, followed by excision of the protein band of interest. Protein bands were analyzed by Proteinworks Co (Seoul, Korea).

The protein band in the gel was digested with trypsin and the resulting peptide was analyzed using a Poros reversed phase R2 column (PerSeptive Biosystems, Framingham, Mass., USA). The isolated peptides were investigated using the electrospray ionization time of a flight mass spectrometer/mass spectrometer (4700 MALDI-TOF/TOF, Applied Biosystems, Thermo Fisher Scientific). Peptide sequences were analyzed using the database of the National Center for Biotechnology Information (NCBI, http://www.ncbi.nlm.nih.gov).

### Example 6: Western Blotting and Immunoprecipitation

Uninfected Vero cell lysates and MERS-CoV-infected cell lysates were prepared with cell lysis buffer (20 mM Tris HCl pH 8.0.5 mM EDTA, 150 mM NaCl, 100 mM NaF, 2 mM Na₃VO₄, 1% NP-40) and after centrifugation at 14,000 rpm for 20 mM at 4° C., separation was performed on a 4-12% Bis-Tris gradient gel (Thermo Fisher Scientific).

The isolated protein was transferred to a nitrocellulose membrane and then the membrane was incubated with anti-S mAb, anti-M mAb, anti-N Ab or anti-β actin antibody overnight at 4° C. After incubating the membrane with horseradish peroxidase-conjugated secondary antibody, the immunoreactive band was reacted with enhanced chemiluminescence (ECL) reagent (Thermo Fisher Scientific).

To perform the binding properties of each MERS-CoV protein, co-immunoprecipitation analysis was performed with each MERS-CoV protein as described above. Coimmunoprecipitated proteins were identified by Western blotting using anti-S mAbs, anti-M mAbs or anti-N Abs.

### Example 7: Analysis of the Interaction Between MERS-CoV Spike CD Peptide and N Protein

MERS-CoV-infected cell lysates was incubated with one of the following biotinylated peptides at 4° C. for 2 hours,

Spike CD-whole-biotin,

Spike CD-F-Biotin,

Spike CD-M-biotin and

Spike CD-B-Biotin

After incubation, after addition of streptavidin-agarose (Thermo Fisher Scientific), immune complexes were collected by centrifugation.

Immune complexes were separated on 10% SDS-PAGE and then analyzed by Western blotting using anti-N Ab. The band density was analyzed by the program of Quantity One (Bio-Rad, Hercules, Calif., USA).

To determine the major regions of Spike-CD involved in the interaction with the N protein, MERS-CoV infected cell lysates were incubated with the respective peptides of Spike CD-F, Spike CD-M and Spike CD-B at 4° C. After incubation for 1 hour, Spike CD-Biotin was added to each sample and then incubated at 4° C. for 2 hours. The interaction of the N protein with Spike CD-biotin was determined by immunoprecipitation with streptavidin-agarose as described above.

### Example 8: Cell Permeation of MERS-CoV Spike CD Peptides

Vero cells (5×10⁴) were seeded on cover glasses in 12 well plates. One day later, the cells were incubated with R-Spike CD-Biotin in a 5% CO₂ incubator at 37° C. for 30 min.

After fixing the cells with 4% paraformaldehyde, the cells were permeabilized with PBST containing 1% BSA. Alexa flour-488-attached streptavidin (Jackson ImmunoResearch Laboratories Inc.) was added and incubated for 1 hour, then the samples were washed with PBST. Nuclei were stained by addition of Hoechst 33258 (Thermo Fisher Scientific). The slides were analyzed by Carl Zeiss LSM710 (Carl Zeiss Co. Ltd. Oberkochen, Germany).

### Example 9: Analysis of MERS-CoV S Protein Expression Using Confocal Microscopy

Vero cells (5×10⁴) were cultured overnight on cover glass in 12 well plates and infected with MERS-CoV (0.1 MOI) with PBS or R-Spike CD in serum-free medium.

After 48 h, cells were fixed and then permeabilized with PBST containing 1% BSA. Permeabilized cells were incubated with anti-S mAb for 2 h. Cells were washed with PBST containing 1% BSA and then incubated with Alexa Flour 488-conjugated goat anti-mouse IgG antibody (Thermo Fisher Scientific) for 1 hour. Nuclei were stained with Hoechst 33258 and then slides were investigated with Carl Zeiss LSM710.

### Example 10: Plaque Formation Assay

Vero cells (6×10⁵ cells/well) were plated on 6-well plates (Corning, N.Y., USA) and incubated for 12 hours. MERS-CoV (200 pfu) was mixed with R-Spike-CD or R-CP-1 serially diluted 2-fold in PBS.

A mixture of MERS-CoV and peptide was added to Vero cells and incubated for 1 hour. After incubation, the supernatant was removed, and the plate was replenished with 3 ml of DMEM/F12 medium (Thermo Fisher Scientific) containing 0.6% Oxoid agar. Cells were stained with crystal violet after 4 days, plaque counts were counted and compared to control samples treated with virus only.

### Example 11: Confirmation of SARS-CoV-2 Spike CD Peptide Inhibition of SARS-CoV-2 Production

Vero cells (2×10⁵ cells/well) were plated on 24-well plates (Corning, N.Y., USA) and incubated for 12 hours. Cells were washed with PBS and then infected with SARS-CoV-2 (0.1 MOI) in a 5% CO₂ incubator at 37° C. for 1 hour.

After incubation, the supernatant was removed, and the plates were replenished with DMEM containing 2% FBS. After incubation for 6 h, 5 μM of the cell-penetrating spike CD-COVID-19 peptide (R-Spike CD-COVID19), the control cell-penetrating peptide (R-CP-1) and the cell-penetrating MERS-CoV spike CD peptide (R-Spike CD (MERS), was treated respectively. After incubation for 17 hours, viral RNA was isolated from the cell culture medium, and cDNA was prepared. The produced virus was quantified by performing real-time RCR using primers for SARS-CoV-2 RNA-dependent RNA polymerase (RdRP).

### Example 12: Real-Time RT-PCR

RNA isolation from virus in cell culture was performed using QIAamp Viral RNA Mini Kit (Catalog No. 52904, Qiagen, Hilden, Germany), and cDNA was synthesized using Reverse Transcription System kit (Catalog No. A3500, Promega, Madison, Wis., USA).

To quantify SARS-CoV-2 in cell culture, the following primers for the RNA-dependent RNA polymerase (RdRP) gene of SARS-CoV-2 were synthesized. [Reference, Jeong-Min Kim et al., Identification of Coronavirus Isolated from a Patient in Korea with COVID-19. Osong Public Health Res Perspect. 2020 February; 11(1): 3-7];

Forward primer,
(SEQ ID NO: 11)
5-GTGAAATGGTCATGTGTGGCGG′
Reverse primer,
(SEQ ID NO: 12)
5′-CAAATGTTAAAAACACTATTAGCATA-3′,
TaqMan ® Probe,
(SEQ ID NO: 13)
5′-FAM-CAGGTGGAACCTCATCAGGAGATGC-TAMRA-3′

Primers and TaqMan® Probe sequences were synthesized by Genotech (Daejeon, South Korea). 10 μL of GoTaq®Probe qPCR Master Mix (Promega, Madison, Wis., USA) was added to 10 μL of forward and reverse primers (125 nM each) and TaqMan®Probe (250 nM) mixture, and 1 μL of cDNA solution was added. The mixture was heated at 95° C. for 5 minutes, followed by 45 cycles of PCR at 95° C. for 15 sec and 60° C. for 1 minute each. The copy number of the RdRP gene was calculated by obtaining a standard curve from the cDNA of the RdRP gene.

### Example 13: HCoV-OC43 Infection and S Protein Binding Protein Analysis

Vero cells (6×10⁵ cells/well) were cultured in 6-well plates for 12 h. HCoV-OC43 (0.1 MOI) was inoculated into Vero cells in PBS and incubated for 1 hour at 37° C. in 5% CO₂ incubator. After incubation, the supernatant was removed and each dish was supplemented with DMEM medium containing 2% FBS, 25 mM HEPES, 100 U/ml penicillin and 100 μg/ml streptomycin.

3 days post infection, HCoV-OC43-infected Vero cells were lysed in cell lysis buffer (10 mM HEPES, 150 mM NaCl, 5 mM EDTA, 100 mM NaF, 2 mM Na₃VO₄, protease inhibitor cocktail and 1% NP-40) at 4° C. for 30 mM. Uninfected Vero cell lysates and HCoV-OC43-infected cell lysates were prepared with cell lysis buffer (20 mM Tris HCl pH 8.0.5 mM EDTA, 150 mM NaCl, 100 mM NaF, 2 mM Na₃VO₄, 1% NP-40) and after centrifugation at 14,000 rpm for 20 min at 4° C., separation was performed on a 4-12% Bis-Tris gradient gel (Thermo Fisher Scientific).

The isolated protein was transferred to a nitrocellulose membrane and then the membrane was incubated with anti-S Ab, anti-N mAb or anti-β actin antibody overnight at 4° C. After incubation of the membrane with horseradish peroxidase-conjugated secondary antibody, the immunoreactive band was reacted with enhanced chemiluminescence (ECL) reagent (Thermo Fisher Scientific).

After immunoprecipitation with anti-S Ab from HCoV-OC43-infected cell lysates, the immune complexes were separated by 4-12% gradient SDS-PAGE, and then co-immunoprecipitated proteins were identified by western blotting using anti-N mAbs.

### Example 14: Analysis of HCoV-OC43 S Protein and N Protein Expression Using Confocal Microscopy

Vero cells (5×10⁴) were cultured overnight on cover glasses in 12 well plates, infected with HCoV-OC43 (0.1 MOI) in PBS for 1 hour, and cultured in DMEM medium containing 2% FBS. After 6 hours, R-Spike CD-OC43 was treated.

After 48 h, cells were fixed and then permeabilized with PBST containing 1% BSA. Permeabilized cells were incubated with anti-S Ab or anti-N mAb for 2 h. Cells were washed with PBST containing 1% BSA and then incubated with Alexa Flour 488-conjugated goat anti-mouse IgG antibody (Thermo Fisher Scientific) or goat anti-rabbit IgG antibody (Thermo Fisher Scientific) for 1 hour. Nuclei were stained with Hoechst 33258 and then slides were examined with Carl Zeiss LSM710.

### Example 15: Confirmation of Inhibition of HCoV-OC43 Production by HCoV-OC43 Spike CD Peptide

Vero cells (2×10⁵ cells/well) were plated on 24-well plates (Corning, N.Y., USA) and incubated for 12 hours. Cells were washed with PBS and then infected with HCoV-OC43 (0.1 MOI) in a 5% CO₂ incubator at 37° C. for 1 hour. After incubation, the supernatant was removed, and the plates were supplemented with DMEM medium containing 2% FBS. After incubation for 6 hours, the cell-permeable spike CD-OC43 peptide (R-Spike CD-OC43) and the spike CD peptide of HCoV-OC43 (Spike CD-OC43) (2 μM each) were respectively treated. After 42 hours of incubation, the virus in the cell culture medium was identified through plaque formation assay.

Vero cells (6×10⁵ cells/well) were plated on 6-well plates (Corning, N.Y., USA) and incubated for 12 hours. after adding the cell-permeable spike CD-OC43 peptide (R-Spike CD-OC43) and the spike CD peptide of HCoV-OC43 (Spike CD-OC43) (2 μM each) to Vero cells incubated for 1 hour. After incubation, the supernatant was removed, and the plate was replenished with 3 ml of DMEM/F12 medium (Thermo Fisher Scientific) containing 0.6% Oxoid agar. Cells were stained with crystal violet after 5 days, plaque counts were counted and compared to control samples treated with virus only.

1. A peptide selected from the group consisting of SEQ ID NO: 1, SEQ ID NO: 6 and SEQ ID NO: 8 that binds to the coronavirus N-protein.

2. The peptide according to claim 1, wherein the peptide is the peptide of SEQ ID NO: 4.

3. The peptide according to claim 1, wherein the coronavirus is Severe acute respiratory syndrome coronavirus, Middle East respiratory syndrome coronavirus, Severe acute respiratory syndrome coronavirus 2, or Human coronavirus OC43 (HCoV-OC43).

4. The peptide according to claim 1, wherein the peptide further comprises a peptide for cell penetration.

5. The peptide according to claim 4, wherein the peptide for cell penetration is a peptide selected from the group consisting of HIV Tat peptide; Pep-1 peptide; oligo-lysine; oligoarginine; and a mixed peptide of oligo-lysine and oligoarginine.

6. A composition for treatment of a coronavirus comprising a coronavirus-derived spike protein or a spike protein fragment thereof as an active ingredient.

7. The composition according to claim 6, wherein the spike protein fragment is a domain after transmembrane of the spike protein.

8. The composition according to claim 6, wherein the coronavirus-derived spike protein or spike protein fragment thereof is a protein of SEQ ID NO: 1, SEQ ID NO: 4, SEQ ID NO: 6, SEQ ID NO: 7, or SEQ ID NO: 8 or a fragment thereof.

9. The composition according to claim 6, wherein the coronavirus is Severe acute respiratory syndrome coronavirus, Middle East respiratory syndrome coronavirus, Severe acute respiratory syndrome coronavirus 2, or Human coronavirus OC43 (HCoV-OC43).

10. The composition according to claim 6, wherein the peptide further comprises a peptide for cell penetration.

11. The composition according to claim 10, wherein the peptide for cell penetration is a peptide selected from the group consisting of HIV Tat peptide; Pep-1 peptide; oligo-lysine; oligoarginine; and a mixed peptide of oligo-lysine and oligoarginine.

12. A composition for binding to a coronavirus N-protein comprising a coronavirus-derived spike protein or a spike protein fragment thereof as an active ingredient.

13. The composition according to claim 12, wherein the spike protein fragment is a domain after transmembrane of the spike protein.

14. The composition according to claim 12, wherein the coronavirus-derived spike protein or spike protein fragment thereof is a protein of SEQ ID NO: 1, SEQ ID NO: 4, SEQ ID NO: 6, SEQ ID NO: 7, or SEQ ID NO: 8 or a fragment thereof.

15. The composition according to claim 12, wherein the coronavirus is Severe acute respiratory syndrome coronavirus, Middle East respiratory syndrome coronavirus, Severe acute respiratory syndrome coronavirus 2, or Human coronavirus OC43 (HCoV-OC43).

16. The composition according to claim 12, wherein the peptide further comprises a peptide for cell penetration.

17. The composition according to claim 16, wherein the peptide for cell penetration is a peptide selected from the group consisting of HIV Tat peptide; Pep-1 peptide; oligo-lysine; oligoarginine; and a mixed peptide of oligo-lysine and oligoarginine.


SEQUENCE LISTING (13 sequences)

SEQ ID NO: 1 - protein, 16 aa, Artificial Sequence
  CD-F
TGCGTNCMGKLKCNRC

SEQ ID NO: 2 - protein, 16 aa, Artificial Sequence
  CD-M
KLKCNRCCDRYEEYDL

SEQ ID NO: 3 - protein, 16 aa, Artificial Sequence
  CD-B
DRYEEYDLEPHKVHVH

SEQ ID NO: 4 - protein, 33 aa, Artificial Sequence
  CD
TGCGTNCMGKLKCNRCCDRYEEYDLEPHKVHVH

SEQ ID NO: 5 - protein, 24 aa, Artificial Sequence
  Control
RRRRRRRRRAQARRKNYGQLDIFP

SEQ ID NO: 6 - protein, 40 aa, Artificial Sequence
  COVID-19
LCCMTSCCSCLKGCCSCGSCCKFDEDDSEPVLKGVKLHYT

SEQ ID NO: 7 - protein, 40 aa, Artificial Sequence
  CD-SARS-CoV
LCCMTSCCSCLKGACSCGSCCKFDEDDSEPVLKGVKLHYT

SEQ ID NO: 8 - protein, 34 aa, Artificial Sequence
  HCoV-OC43
CCTGCGTSCFKKCGGCCDDYTGYQELVIKTSHDD

SEQ ID NO: 9 - protein, 25 aa, Artificial Sequence
  Spike-492
TKPLKYSYINKCSRLLSDDRTEVPQ

SEQ ID NO: 10 - protein, 25 aa, Artificial Sequence
  MERS-M158
CDYDRLPNEVTVAKPNVLIALKMVK

SEQ ID NO: 11 - DNA, 22 bp, Artificial Sequence
  primer
gtgaaatggtcatgtgtggcgg

SEQ ID NO: 12 - DNA, 26 bp, Artificial Sequence
  primer
caaatgttaaaaacactattagcata

SEQ ID NO: 13 - DNA, 25 bp, Artificial Sequence
  probe
caggtggaacctcatcaggagatgc